(19) United States
(12) Patent Application Publication
(10) Pub. No.: US 2022/0135497 A1
(43) Pub. Date: May 5, 2022

(54) PROCESSES FOR PRODUCING AROMATIC AND OLEFINIC COMPOUNDS

(71) Applicant: SABIC Global Technologies B.V., Bergen op Zoom (NL)

(72) Inventors: Travis CONANT, Sugar Land, TX (US); Dustin FICKEL, Sugar Land, TX (US); Raul VELASCO PELAEZ, Sugar Land, TX (US); Joseph William SCHROER, Sugar Land, TX (US)

(73) Assignee: SABIC Global Technologies B.V., Bergen op Zoom (NL)

(21) Appl. No.: 17/310,228

(22) PCT Filed: Jan. 31, 2019
(86) PCT No.: PCT/US2019/016039

§ 371 Date: Jul. 27, 2021

(51) Int. Cl. C07C 2/66; C07C 5/41; C07C 4/02

(52) U.S. Cl. CPC C07C 2/66; C07C 4/025; C07C 5/41

(57) ABSTRACT

Disclosed are systems and processes to produce aromatic and olefinic compounds by aromatization and thermal cracking of hydrocarbons.

### CROSS REFERENCE TO RELATED APPLICATIONS

None.

### BACKGROUND OF THE INVENTION

### A. Field of the Invention

The invention generally concerns systems and processes for producing aromatic and olefinic compounds. In particular, the invention concerns system and processes for producing C6 aromatic compounds with high selectivity.

### B. Description of Related Art

Aromatic hydrocarbons and olefins are important petrochemicals products with a continuously growing demand. Current naphtha cracker integration schemes typically result in approximately 10-12% wt. % total yield (wt./wt.) of C6 to C8 aromatics such as benzene, toluene and xylenes and approximately 18-20 wt. % carbon loss to methane. The yield towards aromatics is typically limited by gas phase thermal cracking yields within the naphtha cracker.

There have been many attempts to improve the yield of aromatics. By way of example, U.S. Pat. No. 7,304,195 to Choi et al. describes using multiple separation steps to remove C6 aromatic hydrocarbons from non-aromatic hydrocarbons and C7 and higher hydrocarbons to improve the yield of C6 hydrocarbons. The non-aromatic hydrocarbons rich in liquefied petroleum gas are subject to a hydrocracking reaction to gaseous products. The C7 and higher hydrocarbons can be subjected to aromatization conditions to produce additional hydrocarbons. In another example U.S. Patent Application Publ. No. 20170144948 to Stevenson et al. describes separating a feed stream into a C6 hydrocarbon feed stream and subjecting the C6 hydrocarbon feed stream to aromatization conditions and separating the aromatic hydrocarbons from the reaction stream. The separated stream is then subjected to hydrocracking conditions to remove trace benzene co-boilers to improve the purity of the produced benzene. These processes suffer from multiple processing steps and require the use of hydrogen. Hydrogen is an expensive resource, which increases the overall capital cost of the process.

While many attempts to optimize the yield of benzene from hydrocarbon feed streams has been made, there is still a need for system and processes for producing aromatic hydrocarbons with high selectivity.

### SUMMARY OF THE INVENTION

A discovery has been made that provides a solution to the low total yield of C6 to C8 aromatic hydrocarbons and high carbon loss to methane obtained from thermal cracking of hydrocarbon mixture (e.g., shale oil condensate, naphtha, and the like). The solution is premised on separating a hydrocarbon mixture (e.g., shale oil condensate, naphtha and like) into C5− hydrocarbons, C6 to C8 hydrocarbons and/or C9+ hydrocarbons. The separated C6 to C8 hydrocarbons can be subjected to aromatizing conditions to produce C6 to C8 aromatic hydrocarbons with at least 90% selectivity. A portion of the unreacted C6 to C8 hydrocarbons from the aromatization reaction can be thermally cracked to produce olefins, pyrolysis oil, pyrolysis gas, or a combination thereof.

In one aspect of the present invention, a process to produce aromatic and olefinic compounds is described. The process can include steps (a) and (b). In step (a), a C6 to C8 hydrocarbons feed stream can be contacted with an aromatization catalyst under conditions suitable to aromatize at least a portion of the C6 to C8 hydrocarbons. A crude products stream comprising C6 to C8 aromatic hydrocarbons and unreacted C6 to C8 hydrocarbons can be produced. The crude product stream can be separated into a C6 to C8 aromatic hydrocarbons stream and an unreacted C6 to C8 hydrocarbons stream. In step (b), olefins, pyrolysis oil, pyrolysis gas, or a combination thereof can be produced by thermal cracking of at least a portion of the unreacted C6 to C8 hydrocarbons stream and/or at least a portion of the unreacted C6 to C8 hydrocarbons stream can be recycled to step (a) to increase production of C6 to C8 aromatic hydrocarbons. Further, C6 aromatic hydrocarbons, C7 aromatic hydrocarbons, and/or C8 aromatic hydrocarbons can be recovered from the C6 to C8 aromatic hydrocarbons stream. The aromatization step (a) conditions can include a temperature of 450 to 650° C., a pressure of 0.03 to 2.17 MPa, and/or a weight hour space velocity (WHSV) of 1 to 100 h⁻¹. The thermal cracking step (b) conditions can include a temperature of 750 to 900° C., a pressure of 0.1 to 0.3 MPa, and/or a and residence times of 50 to 1000 milliseconds. In some aspects, the C6 to C8 hydrocarbons feed stream of step (a) can include linear C6 to C8 hydrocarbons. In some aspects, the C6 to C8 hydrocarbons feed stream of step (a) can include 30 to 99 wt. % of C6 hydrocarbons. In some embodiments, the C6 to C8 hydrocarbons feed stream of step (a) can include 50 to 70 wt. % of C6 hydrocarbons, 20 to 30 wt. % of C7 hydrocarbons, and 5 to 15 wt. % of C8 hydrocarbons. In some aspects, the C6 to C8 hydrocarbons feed stream of step (a) can be obtained from shale oil condensate, naphtha or both. In some aspects, the selectivity for C6 to C8 aromatic hydrocarbons produced in step (a) is at least 90% and selectivity to methane produced in step (a) is less than 5%.

In some embodiments, prior to step (a) a C4+ hydrocarbons stream (e.g., a naphtha or shale oil condensate, etc.) can be separated into a C5− hydrocarbons stream, the C6 to C8 hydrocarbons feed stream of step (a), and a C9+ hydrocarbons stream. The C5− hydrocarbons stream can be provided to step (b) and by cracking of C5− hydrocarbons additional olefins, pyrolysis oil (C11+material), pyrolysis gas (C5 to C10 hydrocarbons), or combinations thereof can be produced.

In some embodiments, a crude hydrocarbon stream can include additional C6 to C8 hydrocarbons, optional unreacted C9+ hydrocarbons, and optional C1 to C4 hydrocarbons. The optional C1 to C4 hydrocarbons can be produced by hydrocracking of the C9+ hydrocarbons stream. The crude hydrocarbon stream can be separated into an additional C6 to C8 hydrocarbons stream, an optional unreacted C9+ hydrocarbons stream if the crude hydrocarbon stream includes unreacted C9+ hydrocarbons, and an optional C1 to C4 hydrocarbons stream if the crude hydrocarbon stream includes unreacted C1 to C4 hydrocarbons. The additional C6 to C8 hydrocarbons stream can be provided to step (a). The optional unreacted C9+ hydrocarbons stream can be provided to step (b).

The crude product stream produced in step (a) can also include gaseous C1 to C4 hydrocarbons. In some aspects, a gaseous C1 to C4 hydrocarbons stream can be separated from the crude product stream and the separated gaseous C1 to C4 hydrocarbons stream and/or the optional C1 to C4 hydrocarbons stream can be provided to a light gas aromatization unit, to a thermal cracking unit, to a catalytic dehydrogenation unit, or a combination thereof.

The pyrolysis gas (C5 to C10 hydrocarbons) produced in step (b) can include C5 to C10 olefins, C5 to C10 paraffinic compounds, and/or C6 to C10 aromatic compounds. In some aspects, a C6 to C8 nonaromatic hydrocarbons stream can be separated from the pyrolysis gas and at least a portion of the C6 to C8 nonaromatic hydrocarbons stream can be provided to step (a).

The pyrolysis gas can be subjected to a hydrotreating step and a fractionation step, and an additional C5− hydrocarbons stream, a third C6 to C8 hydrocarbons stream, and an additional C9+ hydrocarbons stream can be produced. The third C6 to C8 hydrocarbons stream can be subjected to an extraction process and an additional C6 to C8 aromatic hydrocarbons stream and a C6 to C8 nonaromatic hydrocarbons stream can be produced. A portion of the C6 to C8 nonaromatic hydrocarbons stream can be provided to step (a) to produce additional aromatic hydrocarbons. At least a portion of the additional C5− hydrocarbons stream and at least a portion of the C6 to C8 nonaromatic hydrocarbons stream can be recycled to the fractionation step.

In the context of the present invention twenty embodiments are described. Embodiment 1 is a process to produce aromatic and olefinic compounds, the process comprising: (a) contacting a C6 to C8 hydrocarbons feed stream with an aromatization catalyst under conditions suitable to aromatize at least a portion of the C6 to C8 hydrocarbons and produce a crude products stream comprising C6 to C8 aromatic hydrocarbons and unreacted C6 to C8 hydrocarbons; and (b) thermally cracking at least a portion of the unreacted C6 to C8 hydrocarbons to produce olefins, pyrolysis oil, pyrolysis gas, or a combination thereof, and/or recycling at least a portion of the unreacted C6 to C8 hydrocarbons to step (a) to increase production of C6 to C8 aromatic hydrocarbons. Embodiment 2 is the process of embodiment 1, wherein the C6 to C8 hydrocarbons feed stream comprises 30 to 99 wt. % C6 hydrocarbons. Embodiment 3 is the process of embodiment 2, wherein the C6 to C8 hydrocarbons feed stream comprises 50 to 70 wt. % C6 hydrocarbons, 20 to 30% C7 hydrocarbons, and 5 to 15% C8 hydrocarbons. Embodiment 4 is the process of any one of embodiments 1 to 3, wherein the process further comprises: (i) separating the crude product stream from step (a) into a C6 to C8 aromatic hydrocarbons product stream and an unreacted C6 to C8 hydrocarbons stream; and (ii) recovering C6 aromatic hydrocarbons, C7 aromatic hydrocarbons, and/or C8 aromatic hydrocarbons from the C6 to C8 aromatic hydrocarbons product stream. Embodiment 5 is the process of embodiment 4, further comprising recycling at least a portion of the unreacted C6 to C8 hydrocarbons stream to step (a). Embodiment 6 is the process of any one of embodiments 4 to 5, further comprising cracking at least a portion of the unreacted C6 to C8 hydrocarbons stream to produce olefins, pyrolysis oil, pyrolysis gas or a combination thereof. Embodiment 7 is the process of any one of embodiments 1 to 6, wherein the selectivity for C6 to C8 aromatic hydrocarbons produced from the step (a) C6 to C8 hydrocarbons feed stream is at least 90% and selectivity to methane produced in step (a) is less than 5%. Embodiment 8 is the process of any one of embodiments 1 to 7, further comprising prior to step (a): separating a C4+ hydrocarbons stream into a C5− hydrocarbons stream, the C6 to C8 hydrocarbons feed stream of step (a), and a C9+ hydrocarbons stream; and providing the C5− hydrocarbons stream to step (b) and cracking the C5− hydrocarbons to produce additional pyrolysis oil, pyrolysis gas, olefins, or combinations thereof. Embodiment 9 is the process of embodiment 8, further comprising hydrocracking the C9+ hydrocarbons stream under conditions suitable to produce a crude hydrocarbon stream comprising additional C6 to C8 hydrocarbons, optional unreacted C9+ hydrocarbons, and optional C1 to C4 hydrocarbons. Embodiment 10 is the process of embodiment 9, further comprising separating the crude hydrocarbon stream into an additional C6 to C8 hydrocarbons product stream, an optional unreacted C9+ hydrocarbons stream, and an optional C1 to C4 hydrocarbons stream and providing the additional C6 to C8 hydrocarbons stream to step (a). Embodiment 11 is the process of embodiment 10, further comprising cracking at least a portion of the step (a) unreacted C6 to C8 hydrocarbons, the additional C6 to C8 hydrocarbons, the unreacted C9+ hydrocarbons, the C5− hydrocarbons, or any combination thereof. Embodiment 12 is the process of any one of embodiments 4 to 11, wherein the crude product stream further comprises gaseous C1 to C4 hydrocarbons and the process further comprises separating a C1 to C4 hydrocarbons stream from the crude product stream and providing the separated C1 to C4 hydrocarbons stream and/or the optional C1 to C4 hydrocarbons stream to an optional light gas aromatization unit, an thermal cracking unit, or a furnace. Embodiment 13 is the process of any one of embodiments 1 to 12, wherein the pyrolysis gas comprises C5 to C10 olefins, C5 to C10 paraffinic compounds and C5 to C10 aromatic compounds, and the process further comprises separating a C6 to C8 nonaromatic hydrocarbons stream from the pyrolysis gas and providing at least a portion of the C6 to C8 nonaromatic hydrocarbons stream to step (a) and contacting the C6 to C8 nonaromatic hydrocarbons with the aromatization catalyst to produce additional C6 to C8 aromatic hydrocarbons. Embodiment 14 is the process of embodiment 13, further comprising subjecting the pyrolysis gas to: (iii) hydrotreating; and (iv) fractionation to produce an additional C5− hydrocarbons stream, a third C6 to C8 hydrocarbons stream, and an additional C9+ hydrocarbons stream. Embodiment 15 is the process of embodiment 13, further comprising: (v) subjecting the third C6 to C8 hydrocarbons stream to an extraction process to produce an additional C6 to C8 aromatic hydrocarbons stream and a C6 to C8 nonaromatic hydrocarbons stream; and (vi) providing a portion of the C6 to C8 nonaromatic hydrocarbons stream to step (a) and producing additional aromatic hydrocarbons. Embodiment 16 is the process of any one of embodiments 14 to 15, wherein at least one of steps (iii) to (vi) are processed in a step (a) aromatization unit. Embodiment 17 is the process of embodiment 1, wherein the C6 to C8 hydrocarbons feed stream is obtained from shale oil condensate, naphtha or both. Embodiment 18 is the process of any one of embodiments 1 to 17, wherein the aromatization step (a) conditions comprise a temperature of 450 to 650° C., a pressure of 0.03 to 2.17 MPa, and/or a WHSV of 1 to 100 h⁻¹, and/or the thermal cracking step (b) conditions comprise a temperature of 750 to 900° C., a pressure of 0.1 to 0.3 MPa, and/or a and residence times of 50 to 1000 milliseconds. Embodiment 19 is the process of any one of embodiments 1 to 18, wherein the C6 to C8 hydrocarbons feed stream comprises linear C6 to C8 hydrocarbons.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to other aspects of the invention. It is contemplated that any embodiment or aspect discussed herein can be combined with other embodiments or aspects discussed herein and/or implemented with respect to any method or composition of the invention, and vice versa. Furthermore, compositions and systems of the invention can be used to achieve methods of the invention.

The following includes definitions of various terms and phrases used throughout this specification.

The phrases “thermal cracking of hydrocarbons” or “thermal cracking” refer to heating a hydrocarbon to a temperature sufficient to break a carbon-hydrogen bond and/or a carbon-carbon bond and produce lower molecular weight hydrocarbons from a higher molecular weight hydrocarbon, thus reducing the carbon number of the starting hydrocarbon. Thermal cracking does not include the use of a catalyst.

The phrases “hydrocracking of hydrocarbons” or “hydrocracking cracking” refer to cracking of hydrocarbons in a hydrogen (H₂) rich atmosphere at elevated pressures in the presence of a catalyst. Hydrocracking conditions generally include a temperature of 200° C. to 600° C., elevated pressures of 0.2-20 MPa, space velocities between 0.1-10 h⁻¹. Catalysts used for the hydrocracking process can include transition metals, or metal sulfides on a solid support such as alumina, silica, alumina-silica, magnesia and zeolites.

Cn hydrocarbons refer to hydrocarbons having a carbon number n. Cn+ hydrocarbons refer to hydrocarbons having a carbon number n or higher. Cn− hydrocarbons refer to hydrocarbons having a carbon number n or less. Cn hydrocarbons stream refers to hydrocarbons stream comprising Cn hydrocarbons. By way of example, C4+ hydrocarbons refer to hydrocarbons having a carbon number 4 or higher (e.g. butane, pentane, heptane, etc.). C5− hydrocarbon refers to hydrocarbons having a carbon number 5 or less (e.g. butane, pentane, etc.). C6 to C8 hydrocarbons refer to hydrocarbons having a carbon number 6 to 8 (e.g. hexane, heptane, octane, etc.). C9+ hydrocarbons refer to hydrocarbons having a carbon number 9 or higher (e.g. nonane, decane, etc.). C6 to C8 aromatic hydrocarbons refer to aromatic hydrocarbons having a carbon number 6 to 8 (e.g. benzene, toluene, xylene, etc.). C1 to C4 hydrocarbons refer to hydrocarbons having a carbon number 1 to 4 (e.g. methane, ethane, propane, butane, etc.). C6 to C8 linear hydrocarbons refer to linear hydrocarbons having a carbon number 6 to 8 (e.g. n-hexane, n-heptane, n-octane, etc.). C5 to C10 olefins refer to olefinic hydrocarbons having a carbon number 5 to 10 (e.g. pentene, hexene, heptene, octene, nonene, decene etc.). C5 to C10 paraffins refer to paraffinic hydrocarbons having a carbon number 5 to 10 (pentane, hexane, heptane, octane, nonane, decane etc.).

The terms “about” or “approximately” are defined as being close to as understood by one of ordinary skill in the art. In one non-limiting embodiment, the terms are defined to be within 10%, preferably within 5%, more preferably within 1%, and most preferably within 0.5%.

The terms “wt. %”, “vol. %”, or “mol. %” refers to a weight percentage of a component, a volume percentage of a component, or molar percentage of a component, respectively, based on the total weight, the total volume of material, or total moles, that includes the component. In a non-limiting example, 10 grams of component in 100 grams of the material is 10 wt. % of component.

The term “substantially” and its variations are defined to include ranges within 10%, within 5%, within 1%, or within 0.5%.

The terms “inhibiting” or “reducing” or “preventing” or “avoiding” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The use of the words “a” or “an” when used in conjunction with any of the terms “comprising,” “including,” “containing,” or “having” in the claims, or the specification, may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The process and systems of the present invention can “comprise,” “consist essentially of,” or “consist of” particular ingredients, components, compositions, steps, etc. disclosed throughout the specification. With respect to the transitional phrase “consisting essentially of,” in one non-limiting aspect, a basic and novel characteristic of the processes and the systems of the present invention are their abilities to produce aromatic and olefinic compounds by aromatization and cracking of hydrocarbons with 90% C6 hydrocarbon selectivity.

Other objects, features and advantages of the present invention will become apparent from the following figures, detailed description, and examples. It should be understood, however, that the figures, detailed description, and examples, while indicating specific embodiments of the invention, are given by way of illustration only and are not meant to be limiting. Additionally, it is contemplated that changes and modifications within the spirit and scope of the invention will become apparent to those skilled in the art from this detailed description. In further embodiments, features from specific embodiments may be combined with features from other embodiments. For example, features from one embodiment may be combined with features from any of the other embodiments. In further embodiments, additional features may be added to the specific embodiments described herein.

### BRIEF DESCRIPTION OF THE DRAWINGS

Advantages of the present invention may become apparent to those skilled in the art with the benefit of the following detailed description and upon reference to the accompanying drawings.

FIGS. 1A and 1B are schematics of systems of the present invention to produce aromatic and olefinic compounds.

FIG. 2 is a schematic of another systems of the present invention to produce aromatic and olefinic compounds.

FIG. 3 is schematic of an example of the present invention to produce additional aromatic and olefinic compounds by processing the pyrolysis gas produced using the process of FIG. 1A, FIG. 1B, or FIG. 2.

While the invention is susceptible to various modifications and alternative forms, specific embodiments thereof are shown by way of example in the drawings. The drawings may not be to scale.

### DETAILED DESCRIPTION OF THE INVENTION

A discovery has been made that provides a solution to the current problems associated with low C6 aromatic hydrocarbons selectivity and high methane selectivity obtained by thermal cracking of a hydrocarbon mixture (e.g., shale oil condensate, naphtha and like). The solution is premised on using an aromatization unit in combination with a thermal cracking unit to produce C6 aromatic hydrocarbons, preferably, benzene, in selectivities of greater than 90%.

These and other non-limiting aspects of the present invention are discussed in further detail in the following paragraphs with reference to the figures.

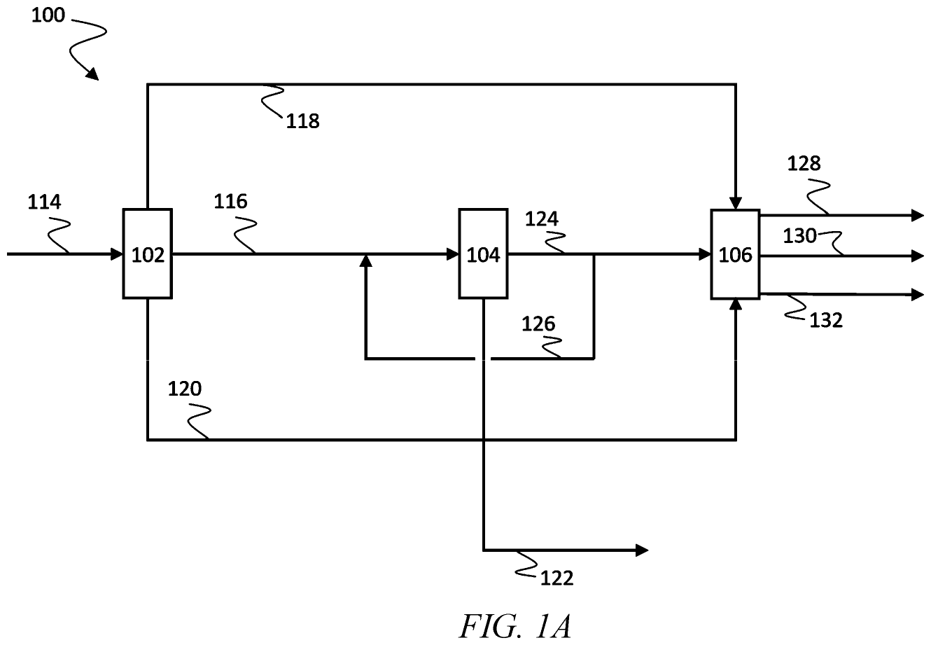

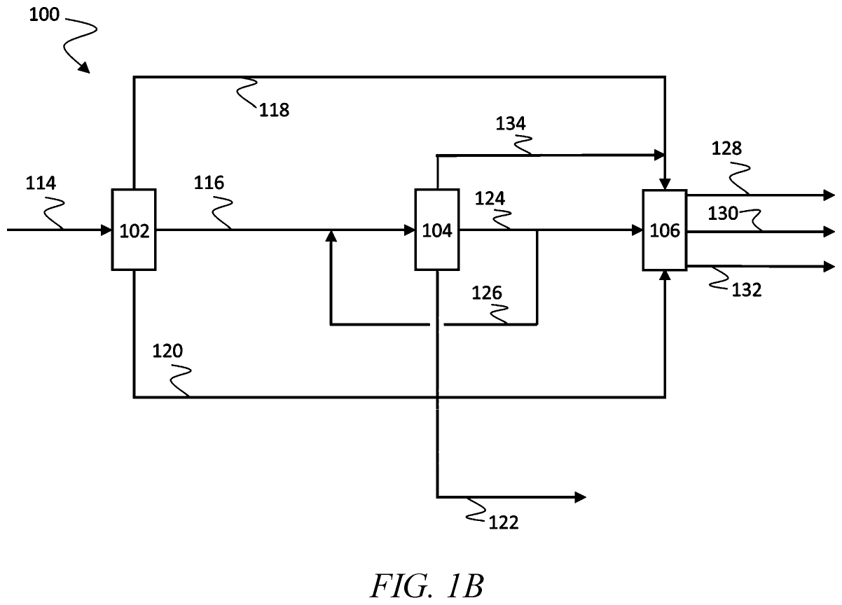

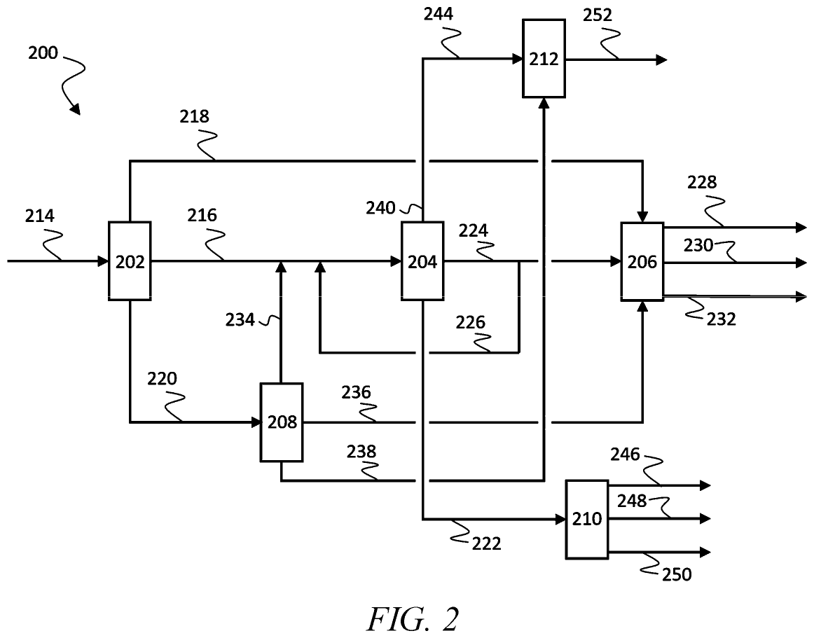

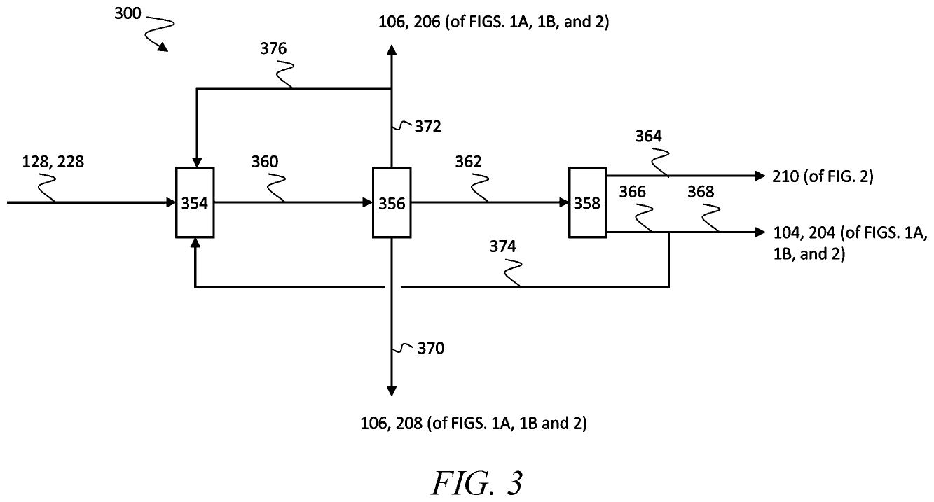

Referring to FIGS. 1A and 1B, system and processes to produce aromatic and olefinic compounds is described. System 100 can include a separation unit 102, a C6+ aromatization unit 104, and a thermal cracking unit 106. A hydrocarbons stream 114 can be fed to the separation unit 102. In the separation unit 102 the hydrocarbon stream 114 can be separated into a C5− hydrocarbons stream 118, a C6 to C8 hydrocarbons stream 116, and a C9+ hydrocarbons stream 120. The C5− hydrocarbons stream 118 and the C9+ hydrocarbons stream 120 can be fed to the thermal cracking unit 106. The C6 to C8 hydrocarbons stream 116 can be fed to the C6+ aromatization unit 104.

Referring to FIGS. 1A and 1B, C6 to C8 hydrocarbons can be contacted with an aromatization catalyst in C6+ aromatization unit 104, under conditions suitable to aromatize at least a portion of the C6 to C8 hydrocarbons and produce a crude product stream that can include C6 to C8 aromatic hydrocarbons and unreacted C6 to C8 hydrocarbons. In C6+ aromatization unit 104, hydrogen can also be produced and separated from the crude product stream (not shown). The hydrogen can be collected, transported and/or provided to other processing units. The crude product stream can be separated into a C6 to C8 aromatic hydrocarbons product stream 122 and an unreacted C6 to C8 hydrocarbons stream 124. The unreacted C6 to C8 hydrocarbons stream 124 can be fed to the thermal cracking unit 106. In some aspects, a portion 126 of the unreacted C6 to C8 hydrocarbons stream 124 can be fed back to the C6+ aromatization unit 104 (not shown) and/or, as shown in FIG. 1, be combined with the C6 to C8 hydrocarbon stream entering the C6+ aromatization unit. C6 aromatic hydrocarbons, C7 aromatic hydrocarbons, and C8 aromatic hydrocarbons can be separated from the C6 to C8 aromatic hydrocarbons product stream 122. In some embodiments, the crude product stream can include C1 to C4 hydrocarbons. Referring to FIG. 1B, system 100 is shown when C1 to C4 hydrocarbons are produced. The crude product stream can be separated into a C6 to C8 aromatic hydrocarbons product stream 122, an unreacted C6 to C8 hydrocarbons stream 124, and a C1 to C4 hydrocarbons stream 134. The C1 to C4 hydrocarbons stream 134 can be combined with the C5− hydrocarbon stream from separation unit 118 and provided to the thermal cracking unit 106. In some embodiments, the C1 to C4 hydrocarbons stream 134 is provided directly to the thermal cracking unit 106. In other instances a portion or all of the C1 to C4 hydrocarbons stream 134 can be provided to a furnace or other processing units (e.g., a naphtha cracking furnace). In the thermal cracking unit 106, by thermal cracking of C5− hydrocarbons, C6 to C8 hydrocarbons, C9+ hydrocarbons or any combination thereof, under suitable condition, pyrolysis gas 128, pyrolysis oil 130, and olefins 132 can be produced. In thermal cracking unit 106, hydrogen can also be produced and separated from the crude product stream (not shown). The hydrogen can be collected, transported and/or provided to other processing units.

Referring to FIG. 2, a system and process to produce aromatic and olefinic compounds is described. System 200 can include a separation unit 202, a C6+ aromatization unit 204, a thermal cracking unit 206, a hydrocracking unit 208, a disproportionation unit 210, and a light gas aromatization unit 212.

A hydrocarbons stream 214 can be fed to the separation unit 202. In the separation unit 202, the hydrocarbon stream 214 can be separated into a C5− hydrocarbons stream 218, a C6 to C8 hydrocarbons stream 216 and a C9+ hydrocarbons stream 220. The C5− hydrocarbons stream 218 can be fed to the thermal cracking unit 206. The C6 to C8 hydrocarbons stream can be fed to the C6+ aromatization unit 204. The C9+ hydrocarbons stream 220 can be fed to the hydrocracking unit 208.

In the C6+ aromatization unit 204, C6 to C8 hydrocarbons can be contacted with an aromatization catalyst under conditions suitable to aromatize at least a portion of the C6 to C8 hydrocarbons and produce a crude product stream that includes C6 to C8 aromatic hydrocarbons, unreacted C6 to C8 hydrocarbons, and C1 to C4 hydrocarbons. The crude product stream can be separated into a C6 to C8 aromatic hydrocarbons stream 222, an unreacted C6 to C8 hydrocarbons stream 224, and a C1 to C4 hydrocarbons stream 240. The unreacted C6 to C8 hydrocarbons stream 224 can be fed to the thermal cracking unit 206. In some aspects, a portion 226 of the unreacted C6 to C8 hydrocarbons stream 224 can be fed back to the C6+ aromatization unit 204 (not shown) and/or, as shown in FIG. 2, be combined with the C6 to C8 aromatic hydrocarbon stream entering the aromatization unit. The C6 to C8 aromatic hydrocarbons stream 222 can be fed to the disproportionation unit 210. The C1 to C4 hydrocarbons stream 240 stream can be feed to the light gas aromatization unit 212. In some embodiments, the C6 to C8 aromatics stream can be separated from the nonaromatic components before feeding to the disproportionation unit. The separation can be performed by any type known in the art, for example liquid extraction by the Sulfolane™ process (UOP, USA), extractive distillation using the Sulfolane™ (UOP) solvent or Morphylane™ (ThyssenKrupp, Germany) solvent, adsorption, and/or combined with distillation. In some embodiments, the C6 to C8 aromatics stream can be further divided, fractionated, and separated as product streams upstream and/or instead of sending to the disproportionation unit.

In the disproportionation unit 210, a C6 aromatic hydrocarbon stream 246, a C7 aromatic hydrocarbon stream 248, and a C8 aromatic hydrocarbon stream 250 can be produced from the C6 to C8 aromatic hydrocarbons stream 222. The disproportionation unit can include reactors/reaction systems for converting one aromatic to another aromatic. For example, trans-alkylation unit can be included to convert toluene into benzene and xylenes, isomerization units can be included to convert ortho- and/or meta-xylene into para-xylene. Hydrodealkylation units can be included to convert toluene, and/or xylenes, and/or ethylbenzene into benzene in the presence of hydrogen. Hydrodealkylation can be performed thermally or catalytically. The disproportionation unit can also include internal recycles and any various sequence for processing aromatics that is known in the art or that could be conceived to be implemented. The disproportionation unit can separate at least a portion of stream 222 and disproportionation unit internal streams into any other combination of product streams (not shown), for example, a purified benzene stream and a stream containing both toluene and C8 aromatic hydrocarbons.

In the hydrocracking unit 208, by hydrocracking of C9+ hydrocarbons under suitable condition a crude hydrocarbon stream comprising C6 to C8 hydrocarbons, optionally unreacted C9+ hydrocarbons, and optionally C1 to C4 hydrocarbons can be produced. The hydrogen generated in the aromatization units and/or thermal cracking units can be used as a hydrogen source in the hydrocracking unit 208. In some embodiments, the conditions are adjusted to produce mostly C1 to C4 hydrocarbons, which can then be used as fuel for other processing units (e.g., naphtha cracking furnace). The crude product stream can be separated into a C6 to C8 hydrocarbons stream 234, an optional unreacted C9+ hydrocarbons stream 236, if the crude hydrocarbon stream includes unreacted C9+ hydrocarbons, and an optional C1 to C4 hydrocarbons stream 238, if the crude hydrocarbon stream includes C1 to C4 hydrocarbons. The C6 to C8 hydrocarbons stream 234 can be fed to the aromatization unit 204 (not shown) and/or, as shown in FIG. 2, be combined with the C6 to C8 hydrocarbon stream entering the aromatization unit. The optional unreacted C9+ hydrocarbons stream 236 can be fed to the thermal cracking unit 206. The optional C1 to C4 hydrocarbons stream 238 can be fed to the light gas aromatization unit 212 and/or thermal cracking unit 106 (not shown). In the light gas aromatization unit 212, C1 to C4 hydrocarbons can be aromatized to produce BTX, optional amounts of light gas, and hydrogen.

In the thermal cracking unit 206, by thermal cracking of C5− hydrocarbons, a portion of the unreacted C6 to C8 hydrocarbons, C9+ hydrocarbons or any combination thereof, under suitable condition, pyrolysis gas 228, pyrolysis oil 230, and olefins 232 can be produced.

The pyrolysis gas streams 128, 228 can include C5 to C10 olefins, C5 to C10 paraffins, and C6 to C10 aromatic compounds. FIG. 3, describes a system and process to produce additional aromatic and olefinic compounds by processing pyrolysis gas. In FIG. 3, the pyrolysis gas processing system 300, can include a hydrotreating unit 354, a fractionation unit 356, and an extraction unit 358. The pyrolysis gas streams 128, 228 can be fed to the hydrotreating unit 354. In the hydrotreating unit 354, by hydrotreating of pyrolysis gas and/or other hydrocarbons under suitable condition, a hydrotreated hydrocarbon stream 360 can be produced. The hydrotreated hydrocarbon stream 360 can be fractionated in the fractionation unit 356, to produce a C6 to C8 hydrocarbons stream 362, a C9+ hydrocarbons stream 370 and a C5− hydrocarbons stream 372. A portion 376 of the C5− hydrocarbons stream 372 can be fed back to the hydrotreating unit 354 and/or the fractionation unit 356 (not shown). In some aspects, the C5− hydrocarbons stream 372 can be fed to the thermal cracking units 106, 206 (of FIGS. 1A, 1B, and 2, respectively). In some aspects, the C9+ hydrocarbons stream 370 can be fed to the thermal cracking 106 unit (of FIG. 1). In some aspects, the C9+ hydrocarbons stream 370 can be fed to hydrocracking unit 208 (of FIG. 2). The C6 to C8 hydrocarbons stream 362 can be fed to the extraction unit 358, and by an extraction process a C6 to C8 aromatic hydrocarbons stream 364 and a C6 to C8 nonaromatic hydrocarbons stream 366 can be obtained. In some aspects, a portion 368 of the nonaromatic hydrocarbons stream 366 can be fed to the C6+ aromatization unit(s) 104, 204 (of FIG. 1, 2 respectively). In some aspects, a portion 374 of the nonaromatic hydrocarbons stream 366 can be fed back to the hydrotreating unit 354 and/or the fractionation unit 356 (not shown). In some aspects, the C6 to C8 aromatic hydrocarbons stream 364 can be fed to the disproportionation unit 210 (of FIG. 2).

In some aspects, the hydrocarbons streams 114, 214 can include C4+ hydrocarbons. In some aspects the hydrocarbons streams 114, 214 are obtained from shale oil condensate, naphtha, or both. Separation of hydrocarbons in the separation unit 102, 202 can be obtained by any suitable methods known in the art e.g., distillation, fractionation, pressure swing adsorption, and the like. In some aspects, the C6 to C8 hydrocarbon stream 116, 216 can include at least any one of, equal to any one of, or between any two of 30%, 40%, 50%, 60%, 70%, 80%, 90% and 99% C6 hydrocarbons. In certain particular aspects, the C6 to C8 hydrocarbon stream 116, 216 can include at least any one of, equal to any one of, or between any two of 50%, 55%, 60%, 65%, and 70% C6 hydrocarbons, at least any one of, equal to any one of, or between any two of 20%, 25%, and 30% C7 hydrocarbons at least any one of, equal to any one of, or between any two of 5%, 10%, and 15% C8 hydrocarbons. In some aspects, the C6 to C8 hydrocarbon stream 116, 216 can include linear C6 to C8 hydrocarbons.

The aromatization reaction condition in the C6+ aromatization units 104, 204 and/or light gas aromatization unit 212 can include a temperature of at least any one of, equal to any one of, or between any two of 450° C., 500° C., 550° C., 600° C. and 650° C., a pressure of at least any one of, equal to any one of, or between any two of 0.03 MPa, 0.2 MPa, 0.4 MPa, 0.6 MPa, 0.8 MPa, 1 MPa, 1.2 MPa, 1.4 MPa, 1.6 MPa, 1.8 MPa, 2 MPa and 2.17 MPa, and/or a WHSV of at least any one of, equal to any one of, or between any two of 1 h⁻¹, 10 h⁻¹, 20 h⁻¹, 30 h⁻¹, 40 h⁻¹, 50 h⁻¹, 60 h⁻¹, 70 h⁻¹, 80 h⁻¹, 90 h⁻¹, and 100 h⁻¹. The aromatization catalyst of the C6+ aromatization unit 104, 204, or the light gas aromatization unit can be any aromatization catalyst known in the art. In some aspects, the aromatization catalyst can also catalyze skeletal isomerization of hydrocarbons, for example, the aromatization catalyst can catalyze in-situ isomerization of iso-hexane to n-hexane and subsequent aromatization of n-hexane to benzene. In some aspects, the aromatization catalyst can include a non-acidic aluminum-silicon-germanium zeolite on which a noble metal has been dispersed. The noble metal can be platinum, palladium, iridium, rhodium and ruthenium. The zeolite can be ZSM-5, ZSM-8, ZSM-11, ZSM-12, ZSM-35, ZSM-38 or any combination thereof. In certain particular aspects, the aromatization catalyst can include highly dispersed platinum on a GeZSM-5 that has been treated with alkali metal(s). The aromatization catalyst can be an aromatization catalyst as described in U.S. Pat. No. 6,784,333 to Juttu et al., and U.S. Pat. No. 7,902,413 to Stevenson et al, which are incorporated herein by reference. For example, the catalyst can be represented as: M[(SiO₂)(XO₂)ₓ(YO₂)_{y}]Z⁺_{y/n} where M is a noble metal, such as platinum, palladium, rhodium, iridium, ruthenium or combinations thereof, X is a tetravalent element, Y is aluminum and, optionally, another trivalent element, Z is a cation or combination of cations with a valence of n, such as H⁺, Na⁺, K⁺, Rb⁺, Cs⁺, Ca²⁺, Mg²⁺, Sr²⁺ or Ba²⁺, and x varies from 0-0.15 and y is 0-0.125. According to the IUPAC recommendations, an example catalyst would be represented as: Cs⁺Pt[Si₉₁Ge₄Al₁O₁₉₂]-MFI or H⁺Pt[Si₉₁Ge₄Al₁O₁₉₂]-MFI. In some aspects, aromatization unit 104, 204 can include, an aromatization reactor, a hydrotreating reactor and a fractionator.

The thermal cracking reaction condition in the thermal cracking unit 106, 206 can include a temperature of at least of any one of, equal to any one of, or between any two of 750° C., 800° C., 850° C., and 900° C., a pressure of at least of any one of, equal to any one of, or between any two of 0.1 MPa, 0.15 MPa, 0.2 MPa, 0.25 MPa, and 0.3 MPa, and/or residence times of at least any one, equal to any one, or between any two of 50 milliseconds, 100 milliseconds, 200 milliseconds, 300 milliseconds, 400 milliseconds, 500 milliseconds, 600 milliseconds, 700 milliseconds, 800 milliseconds, 900 milliseconds, and 1000 milliseconds.

In FIGS. 1-3 the reactors, units and/or zones can include one or more heating and/or cooling devices (e.g., insulation, electrical heaters, jacketed heat exchangers in the wall) or controllers (e.g., computers, flow valves, automated values, etc.) that are necessary to control the reaction temperature and pressure of the reaction mixture. While only one unit or zone is shown, it should be understood that multiple reactors or zones can be housed in one unit or a plurality of reactors housed in one heat transfer unit.

### EXAMPLES

The present invention will be described in greater detail by way of specific examples. The following examples are offered for illustrative purposes only, and are not intended to limit the invention in any manner. Those of skill in the art will readily recognize a variety of noncritical parameters which can be changed or modified to yield essentially the same results.

### Example 1

### Producing Aromatic and Olefinic Compounds from Saudi Light Naphtha (A-180)

Example 1 describes calculations for producing aromatic and olefinic compounds by aromatization and thermal cracking of Saudi Light Naphtha (A-180) using SPYRO® (Technip Benelux BV). In the first calculation, experiment 1, naphtha was fed to a thermal cracker and was thermally cracked. In another calculation, experiment 2, naphtha was separated into a C5− hydrocarbons stream, C6-C8 hydrocarbons stream and a C9+ hydrocarbons stream. The C6-C8 hydrocarbons stream was fed into an aromatization unit and was aromatized. The C5− hydrocarbons stream, and the C9+ hydrocarbons stream was fed to a thermal cracker and was thermally cracked. Data presented in Table 1 shows yield and production differences between the two calculations. The weight percentage yield of the C6 to C8 aromatics increased from 12 to 24% between calculation 1 and calculation 2. Moreover, overall useful product yield (total yield of C2 hydrocarbons, C3 hydrocarbons, C4 hydrocarbons and benzene) was increased by 3% in calculation 2 compared to calculation 1.

TABLE 1

| | | | Difference in |
|---|---|---|---|
| | Calculation 1 | Calculation 2 | production |
| Products | (kta) | (kta) | (MMUSD) |
| CO₂ | 9.9 | 8.4 | (1.5) |
| Hydrogen | 51.7 | 100.8 | 49.1 |
| Methane | 743.6 | 635.3 | (108.2) |
| Ethylene - polymer grade | 1484.7 | 1199.8 | (284.9) |
| Propylene - polymer grade | 712.0 | 603.3 | (108.7) |
| Butadiene | 220.6 | 187.1 | (33.5) |
| MTBE | 180.0 | 171.6 | (8.4) |
| Butene-1 | 55.3 | 45.5 | (9.7) |
| Benzene | 315.7 | 842.4 | 526.7 |
| Toluene | 93.3 | 84.2 | (9.1) |
| C8 aromatics | 18.7 | 17.1 | 1.6 |
| C9 aromatics | 1.2 | 1.1 | (0.1) |
| Full range pyrolysis oil | 113.4 | 103.4 | (10.0) |
| Total | 4000.0 | 4000.0 | 0.0 |

### Example 2

### Reformation of Separated Gas Stream

Example 2 describes calculations for producing aromatic and olefinic compounds from a C6 hydrocarbons stream. Parallel calculations were run. In one calculation, experiment 3, a C6 hydrocarbons steam, from Saudi Light Naphtha (A-180), was fed to a thermal cracking unit and was thermally cracked. In another calculation, experiment 4, a C6 hydrocarbons stream, from Saudi Light Naphtha (A-180), was fed to an aromatization unit and was aromatized. Table 2 shows the wt. % yield of the products obtained in the experiments 3 and 4. The weight percentage yield of useful products (total yield of C2 hydrocarbons, C3 hydrocarbons, C4 hydrocarbons and benzene) was increased from 78% to 90% between experiments 3 and 4. Methane wt. % yield was decreased from 17% to 1% between experiments 3 and 4.

TABLE 2

| | Experiment 3 | Experiment 4 |
|---|---|---|
| Products | (Wt. %) | (Wt. %) |
| CO₂ | 0.2 | 0.01 |
| Hydrogen | 1.3 | 9.01 |
| Methane | 17.1 | 0.88 |
| Ethylene - polymer grade | 49.1 | 1.51 |
| Propylene - polymer grade | 17.3 | 0.60 |
| Butadiene | 5.3 | 0.10 |
| MTBE | 0.5 | 0.24 |
| Butene-1 | 1.6 | 0.03 |
| Benzene | 4.7 | 87.55 |
| Toluene | 1.2 | 0.03 |
| C8 aromatics | 0.2 | 0.01 |
| C9 aromatics | 0.0 | 0.00 |
| Full range pyrolysis oil | 1.5 | 0.03 |
| Total | 100.0 | 100.0 |

Although embodiments of the present application and their advantages have been described in detail, it should be understood that various changes, substitutions and alterations can be made herein without departing from the spirit and scope of the embodiments as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the above disclosure, processes, machines, manufacture, compositions of matter, means, methods, or steps, presently existing or later to be developed that perform substantially the same function or achieve substantially the same result as the corresponding embodiments described herein can be utilized. Accordingly, the appended claims are intended to include within their scope such processes, machines, manufacture, compositions of matter, means, methods, or steps.

1. A process to produce aromatic and olefinic compounds, the process comprising:
(a) contacting a C6 to C8 hydrocarbons feed stream with an aromatization catalyst under conditions suitable to aromatize at least a portion of the C6 to C8 hydrocarbons and produce a crude products stream comprising C6 to C8 aromatic hydrocarbons and unreacted C6 to C8 hydrocarbons; and
(b) thermally cracking at least a portion of the unreacted C6 to C8 hydrocarbons to produce olefins, pyrolysis oil, pyrolysis gas, or a combination thereof, and/or recycling at least a portion of the unreacted C6 to C8 hydrocarbons to step (a) to increase production of C6 to C8 aromatic hydrocarbons;
wherein the selectivity for C6 to C8 aromatic hydrocarbons produced from the step (a) C6 to C8 hydrocarbons feed stream is at least 90% and selectivity to methane produced in step (a) is less than 5%.

2. The process of claim 1, wherein the C6 to C8 hydrocarbons feed stream comprises 30 to 99 wt. % C6 hydrocarbons.

3. The process of claim 2, wherein the C6 to C8 hydrocarbons feed stream comprises 50 to 70 wt. % C6 hydrocarbons, 20 to 30% C7 hydrocarbons, and 5 to 15% C8 hydrocarbons.

4. The process of claim 1, wherein the process further comprises:
(i) separating the crude product stream from step (a) into a C6 to C8 aromatic hydrocarbons product stream and an unreacted C6 to C8 hydrocarbons stream; and
(ii) recovering C6 aromatic hydrocarbons, C7 aromatic hydrocarbons, and/or C8 aromatic hydrocarbons from the C6 to C8 aromatic hydrocarbons product stream.

5. The process of claim 4, further comprising recycling at least a portion of the unreacted C6 to C8 hydrocarbons stream to step (a).

6. The process of claim 4, further comprising cracking at least a portion of the unreacted C6 to C8 hydrocarbons stream to produce olefins, pyrolysis oil, pyrolysis gas or a combination thereof.

7. The process of claim 1, further comprising prior to step (a):
separating a C4+ hydrocarbons stream into a C5− hydrocarbons stream, the C6 to C8 hydrocarbons feed stream of step (a), and a C9+ hydrocarbons stream; and
providing the C5− hydrocarbons stream to step (b) and cracking the C5− hydrocarbons to produce additional pyrolysis oil, pyrolysis gas, olefins, or combinations thereof.

8. The process of claim 2, further comprising prior to step (a):
separating a C4+ hydrocarbons stream into a C5− hydrocarbons stream, the C6 to C8 hydrocarbons feed stream of step (a), and a C9+ hydrocarbons stream; and
providing the C5− hydrocarbons stream to step (b) and cracking the C5− hydrocarbons to produce additional pyrolysis oil, pyrolysis gas, olefins, or combinations thereof.

9. The process of claim 8, further comprising hydrocracking the C9+ hydrocarbons stream under conditions suitable to produce a crude hydrocarbon stream comprising additional C6 to C8 hydrocarbons, optional unreacted C9+ hydrocarbons, and optional C1 to C4 hydrocarbons.

10. The process of claim 9, further comprising separating the crude hydrocarbon stream into an additional C6 to C8 hydrocarbons product stream, an optional unreacted C9+ hydrocarbons stream, and an optional C1 to C4 hydrocarbons stream and providing the additional C6 to C8 hydrocarbons stream to step (a).

11. The process of claim 10, further comprising cracking at least a portion of the step (a) unreacted C6 to C8 hydrocarbons, the additional C6 to C8 hydrocarbons, the unreacted C9+ hydrocarbons, the C5− hydrocarbons, or any combination thereof.

12. The process of claim 10, wherein the crude product stream of step (a) further comprises gaseous C1 to C4 hydrocarbons and the process further comprises separating a C1 to C4 hydrocarbons stream from the crude product stream and optionally providing to a light gas aromatization unit, a thermal cracking unit, or a furnace the separated C1 to C4 hydrocarbons stream from the crude product stream of step (a) and/or the optional C1 to C4 hydrocarbons stream from the crude hydrocarbon stream from the hydrocracked C9+ hydrocarbons stream.

13. The process of claim 1, wherein the pyrolysis gas comprises C5 to C10 olefins, C5 to C10 paraffinic compounds and C5 to C10 aromatic compounds, and the process further comprises separating a C6 to C8 nonaromatic hydrocarbons stream from the pyrolysis gas and providing at least a portion of the C6 to C8 nonaromatic hydrocarbons stream to step (a) and contacting the C6 to C8 nonaromatic hydrocarbons with the aromatization catalyst to produce additional C6 to C8 aromatic hydrocarbons.

14. The process of claim 13, further comprising subjecting the pyrolysis gas to:
(iii) hydrotreating; and
(iv) fractionation to produce an additional C5− hydrocarbons stream, a third C6 to C8 hydrocarbons stream, and an additional C9+ hydrocarbons stream.

15. The process of claim 14, further comprising:
(v) subjecting the third C6 to C8 hydrocarbons stream to an extraction process to produce an additional C6 to C8 aromatic hydrocarbons stream and a C6 to C8 nonaromatic hydrocarbons stream; and
(vi) providing a portion of the C6 to C8 nonaromatic hydrocarbons stream to step (a) and producing additional aromatic hydrocarbons.

16. The process of claim 15, wherein at least one of steps (iii) to (vi) are processed in a step (a) aromatization unit.

17. The process of claim 1, wherein the C6 to C8 hydrocarbons feed stream is obtained from shale oil condensate, naphtha or both.

18. The process of claim 1, wherein the aromatization step (a) conditions comprise a temperature of 450 to 650° C., a pressure of 0.03 to 2.17 MPa, and/or a WHSV of 1 to 100 h⁻¹, and/or the thermal cracking step (b) conditions comprise a temperature of 750 to 900° C., a pressure of 0.1 to 0.3 MPa, and/or a and residence times of 50 to 1000 milliseconds.

19. The process of claim 1, wherein the C6 to C8 hydrocarbons feed stream comprises linear C6 to C8 hydrocarbons.

20. The process of claim 3, further comprising prior to step (a):
separating a C4+ hydrocarbons stream into a C5− hydrocarbons stream, the C6 to C8 hydrocarbons feed stream of step (a), and a C9+ hydrocarbons stream; and
providing the C5− hydrocarbons stream to step (b) and cracking the C5− hydrocarbons to produce additional pyrolysis oil, pyrolysis gas, olefins, or combinations thereof.
